(19) United States
(12) Patent Application Publication
(10) Pub. No.: US 2022/0408147 A1
(43) Pub. Date: Dec. 22, 2022

(54) Realtime Sentiment Analysis and Augmentation for Mental Health Benefits

(71) Applicant: amginE Ink LLC, Los Angeles, CA (US)

(72) Inventor: Brandon Howard, Burbank, CA (US)

(21) Appl. No.: 17/847,129

(22) Filed: Jun. 22, 2022

Related U.S. Application Data
(60) Provisional application No. 63/213,427, filed on Jun. 22, 2021.

(51) Int. Cl. H04N 21/43; H04N 21/466; A61B 5/16; G16H 20/70; G16H 50/30

(52) U.S. Cl. CPC H04N 21/43074; H04N 21/4668; A61B 5/165; G16H 20/70; G16H 50/30

(57) ABSTRACT

Various aspects of the subject technology are designed to analyze a range of inputs (e.g., sound, light, color, words, media, etc.), determine the sub-conscious impact of those inputs on individuals, then recommend and/or augment content capable of improving their current mental health state. Embodiments of the subject technology receive inputs (e.g., data) from information sources or platforms (e.g., smart phones, applications, wearable devices, databases, entered values). Embodiments of the subject technology analyze the received data according to one or more programmed matrices. Embodiments of the subject technology score the subconscious impact. Embodiments of the subject technology compare user data with the content data to make one or more predictions. Embodiments of the subject technology output, display, or provide several solutions for a user to choose from.

### CROSS-REFERENCE TO RELATED APPLICATIONS

This application claims the benefit of, and priority to, U.S. Provisional Patent Application No. 63/213,427, titled “Realtime Sentiment Analysis and Augmentation for Mental Health Benefits,” filed on Jun. 22, 2021, the disclosures of which are incorporated by reference herein in their entirety.

### TECHNICAL FIELD AND BACKGROUND

The technology relates to the field of biofeedback and has certain, specific applications to stress reduction and cognitive behavioral augmentation rooted in color psychology, Hans Selye's general adaptation syndrome regarding how one's body responds to stress, and the science of sound.

### SUMMARY

A methodology and system to recognize stress and its triggers earlier and earlier in their expression in order to prevent, reduce the impact of, or even reverse downstream chronic conditions is desired.

According to one embodiment of the present disclosure, a method is provided for identifying wellness-mediating content, including receiving customer data which includes user profile data and media content items consumed by a user associated with the user profile data, and processing at least a subset of the customer data to determine a multidimensional individual wellness score for the user. The method includes processing the media content items identified in the customer data to determine for each content item a multidimensional content impact score, using a machine learning trained recommendation engine to process the individual wellness score of the user and the content impact scores of the media items consumed by the user to generate a recommended set of wellness-mediating content items. The wellness-mediating content items include at least one of healing sounds, healing colors, healing words, and healing content. The method further includes outputting on an electronic interface for the user the set of recommended wellness-mediating content items generated by the recommendation engine.

According to another embodiment of the present disclosure, a method is provided for causing at least one wellness-mediating content item from a set of wellness-mediating content items to be provided to the user. In another embodiment one or more wellness-mediating content items is output to the user by augmenting a content item with the at least one wellness-mediating content item. In a further embodiment the output can be by: outputting a binaural beat included in the set of recommended wellness-mediating content items, augmenting a content item not in the set of selected recommended wellness-mediating content items by an audio augmentation included in the set of recommended wellness-mediating content items, augmenting a content item not in the set of selected recommended wellness-mediating content items by an color augmentation included in the set of recommended wellness-mediating content items, and playing a piece of media content included in the set of recommended wellness-mediating content items.

According to another embodiment of the present disclosure, a method is provided for identifying wellness-mediating content where the individual content score and the content impact scores each include the dimensions. These dimensions can include, for example, a physical dimension, an emotional dimension, and a mental dimension. For example, individual content scores and the content impact scores can each include a physical dimension, an emotional dimension, and a mental dimension across seven different categories, each category corresponding to one of a color or an audio frequency range.

According to another embodiment of the present disclosure, a method is provided for identifying wellness-mediating content, including electronically receiving user feedback. The feedback can include the identification of at least one item of recommended wellness-mediating content consumed by the user and an impact on the physical, emotional, or mental well-being of the user resulting from the user's consumption. The method can further include applying a machine learning algorithm to adjust the processing of the recommendation engine for the user, a group of users, or all users, based on the feedback. In another embodiment the identification of the impact on the physical, emotional, or mental well-being of the user includes a sentiment change value.

### BRIEF DESCRIPTION OF THE DRAWINGS

FIG. 1 depicts an overview of an exemplary system block diagram as discussed herein, for input, analysis, and output.

FIG. 2 depicts an example color matrix of negative, positive, purpose, and other wellbeing factors and affects.

FIG. 3 depicts an example connection between a database and users to facilitate connections.

FIG. 4 depicts example histograms that can be outputted by the system.

FIGS. 5A-5B depicts example histograms of colors, for further correlation.

FIGS. 6A-6D depict other example histograms of colors, for further correlation and analysis.

FIG. 7 depicts an example U4Ea score that is processed by the system.

FIG. 8 depicts a detailed example of a U4Ea score, including the individual 7 category values.

FIG. 9 depicts an example sentiment prediction/recommendation system.

FIG. 10 depicts an example U4Ea Platform of the technology discussed herein.

FIGS. 11A-11E depict screenshots of various features of the technology discussed herein.

FIGS. 12A-12C depict example user interface augmentations.

FIG. 13A-13C depict additional example user interface augmentations and features.

FIGS. 14A-14E depict additional example user interface augmentations and features.

FIGS. 15A-15F depict additional example user interface augmentations and features.

FIGS. 16A-16F depict additional example user interface augmentations and features.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

U4Ea Scores are used for evaluating wellbeing, whether wellbeing for individuals or the wellbeing impact of content, like media content, where sound, color, and text can be analyzed. It is intended for U4Ea Scores to provide insight and recommendations to individuals in assisting them to finding improved wellbeing through a pursuit toward mental, emotional, and physical balance. It is anticipated that this approach will reduce the impact of chronic illnesses on the individual, as well as economically for payers who will have less chronic related illnesses to treat.

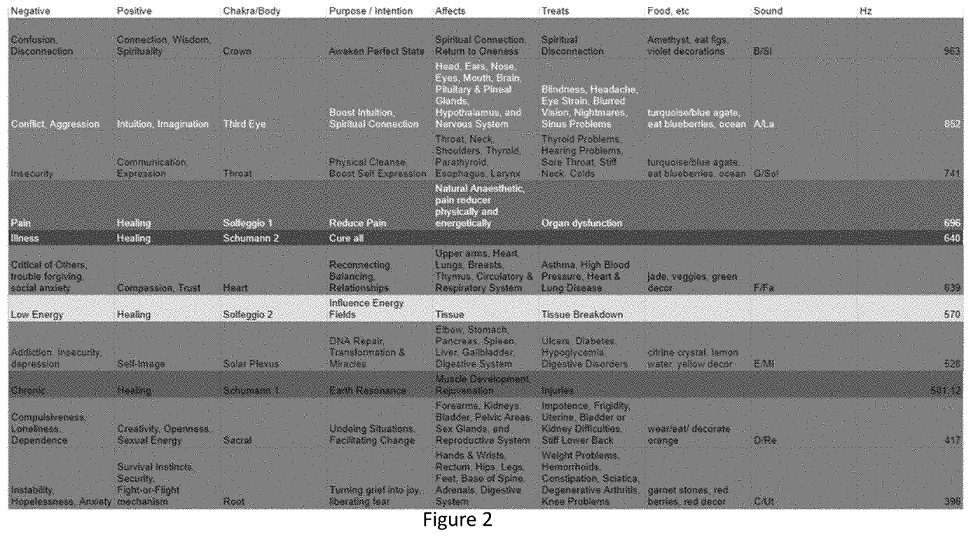

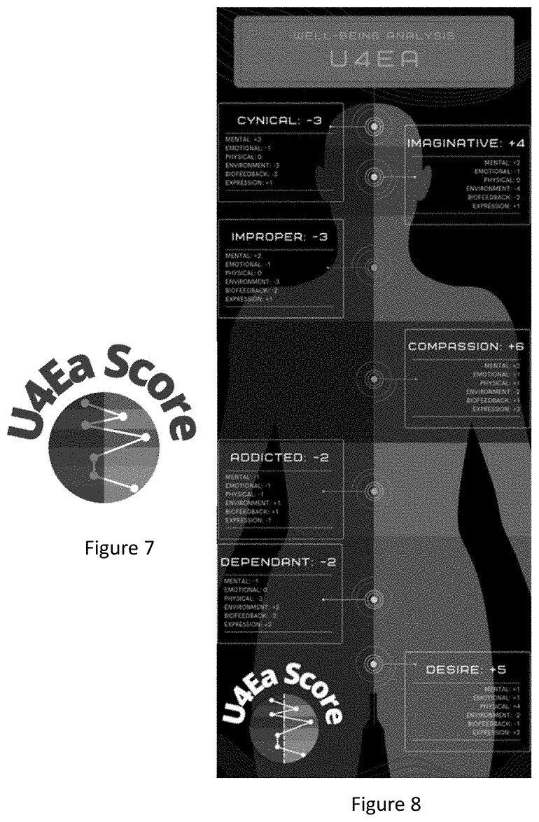

The total U4Ea Score is comprised of seven categories, each pertaining to a correlation of activities, actions, data, or inputs. U4Ea's methodology has concluded correlations between behaviors, the thoughts and emotions associated with their expressions, their associations with the endocrine system, and how the endocrine system responds to related stimulus in the form of sound, color, words, and how these interplay with consciousness. FIG. 2 shows an example elaboration of the correlations associated with each of the seven categories. As data is collected from the customer experience, points are tallied as positive or negative in each category, yielding a composite score reflecting their current areas of balance (see, e.g., FIG. 7 and FIG. 8) (being positioned close to the center line), negativity (to the left of the line), and positive (to the right of the line), with the goal of getting to balanced, or to the customer's desired level. The scores are used to recommend binaural beats, colors, shapes, and a spectrum of third party products and services capable of assisting in shifting scores in a positive direction.

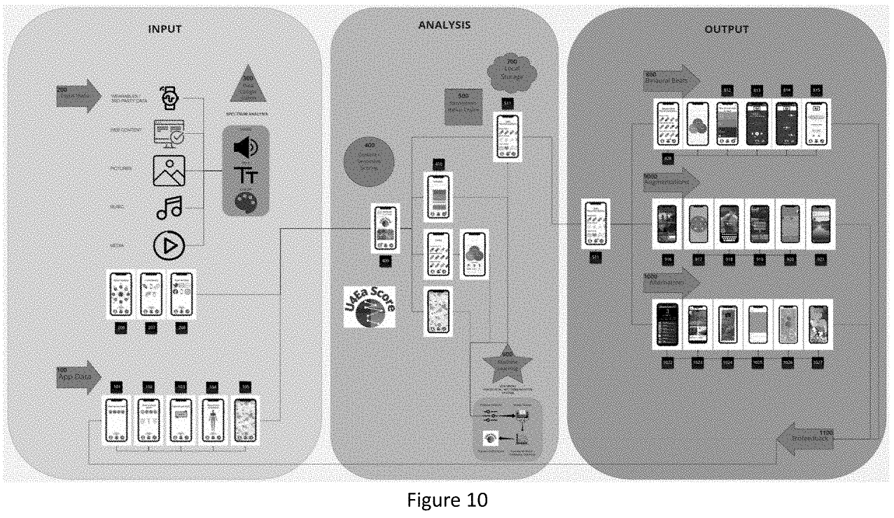

The process to execute this approach to wellbeing involves the U4Ea Platform (with an example shown in FIG. 10). The U4Ea platform executes the process of sentiment recommendations through three phases—Input, Analysis, and Output. The customer will share information with the U4Ea, U4Ea will score the information, tracking the score only, and then U4Ea will output recommendations. U4Ea will further seek feedback on the outcome of the recommendations using a machine learning trained recommendation engine (MLTRE) to improve recommendations. The Input phase below discusses the various ways data is retrieved in order to be analyzed. The Analysis phase discusses how the data is categorized and processed in order to generate a spectrum of recommendations. The Output phase discusses how the binaural beats and related modalities are offered, how they are experienced, and how that information informs the recommendation engine.

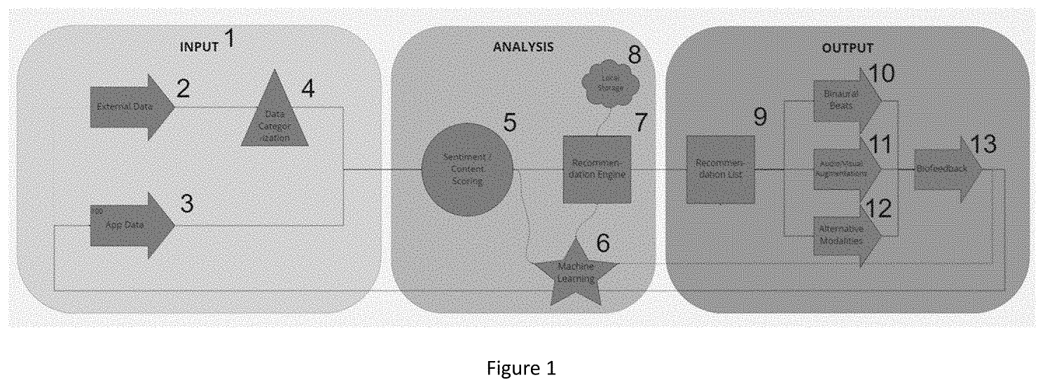

As seen in FIG. 1, Input 1 is utilized to identify wellness-mediating content. Input 1 may be, for example, application data 3. In one embodiment, U4Ea's current Swift coding for iOS is migrating to REACT.js for cross platform accessibility on web, iOS, and Android platforms. Integrating with the Wix platform to establish the primary operating interface, the previously mentioned features, along with content detailed in this document will also be integrated using combinations of HTML, CSS, and JavaScript. Features will include elements of gamification for tracking and feedback purposes. All customer specific tracking features will require customers to have unique log-ins to record activity. Microsoft Azure's SQL database is our methodology for aggregating and storing information.

Gaining information from the customer can happen through customer interaction, by way of permissions to methodologies for tracking information passively, as through wearables or activity tracking on devices or by interacting with question, survey, features built in to U4Ea. The app opens to a Landing Page with U4Ea's logo, listing appropriate legal information, then dissolves to the image in this figure, where the customer is prompted to select either the simple webapp or gamified webapp (through login).

The simple webapp is a trial version of the binaural beats generator. The user is first run through a tutorial using react-joyride, and is prompted to select their feelings, activities, and boosts. Information about the three options is also parsed. After the three options are selected, the disabled attribute on the bottom of the screen is temporarily deleted, and the user is able to press play. After pressing play, a binaural beat object is created, and starts playing.

The gamified webapp is a version of the binaural beats generator that includes user logins and more features. The customer information is stored in a Microsoft Azure database, passwords are encrypted using bcrypt. Once a user logs in, they are able to access more personalized features. Backend code is located in the server folder, and user information is stored in redux store in the client folder after login.

Alternatively, or in addition, as seen in FIG. 1, external data 2 can be received, such as customer data including a user profile data and a plurality of media content items consumed by a user associated with the user profile data. Other potential external data 2 is also contemplated. There are several ways, e.g., at least eight ways, that active and passive sentiment analysis can be offered, to score data and track scores without keeping or storing it: vision tracking, facial recognition, voice analysis, text analysis, scroll tracking, and/or other-app activity; including data from HRV, EEG, and wearables. The ability to configure permissions will be executed through the ‘Settings’ features.

U4Ea intends on capturing data via two methods: in-app interactive surveys (using emojis and U4Ea's “DaVinci” model) and through third party media content.

In one embodiment, app data 3 can be gathered by asking customers, e.g., by prompting for customers to answer the question, “How do you feel?” They can respond using a graphical user interface (GUI), an emoji interface, they can text back a response through a journaling feature, they will be able to interact with a “DaVinci” interface to “point to the pain,” and will be able to link their emotions to a map for planning and tracking their emotional experiences.

U4Ea will analyze media content and information either on a customer's device or via permissions granted by the customer to analyze media on third party platforms. U4ea organizes media into as many as three categories: audio, visual, and text. For example, a song can be organized into two categories, audio for the music and text for the lyrics. A movie would add the visual component where each increment of the movie is analyzed like photo. For example, for data captured from media, the customer will have the opportunity to have their media scored in order to identify the potential subconscious impact of the media, as well as recommending media that can have a positive impact. In one embodiment, U4Ea analyzes sound, color, and text in order to score media content and information.

As seen in FIG. 1, app data 3 can also be utilized to process at least a subset of the customer data to determine a multidimensional individual wellness score for the user.

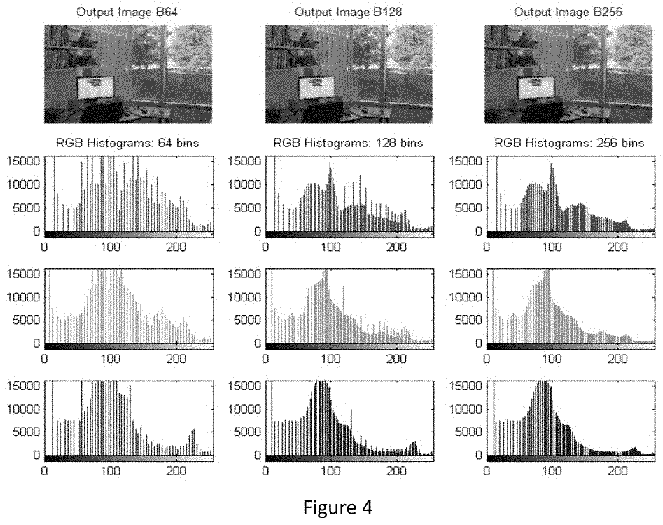

The system can utilize various methods of data categorization. For example, audio files are processed where each note, tone, and/or frequency captured in the audio file is color-correlated. An example color correlation is shown in FIG. 2, as a process of categorizing inputs and mapping them. In one embodiment, U4Ea's methodology blends color psychology with music theory. The emotional responses associated to colors allows the system to mathematically correlate colors to audible tones or frequencies, geometric patterns, words associated with emotions, the endocrine system, foods, and more. This may also be used to create one or more histograms, examples of which are seen in FIG. 4.

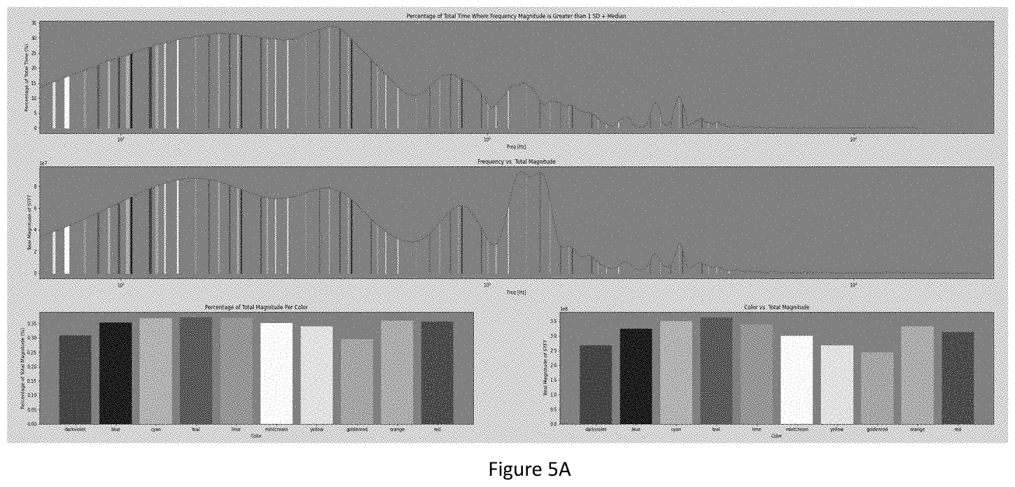

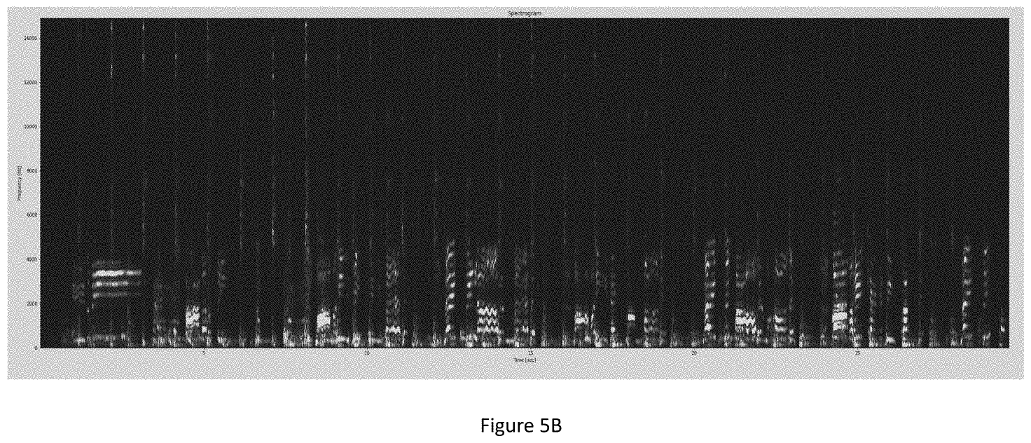

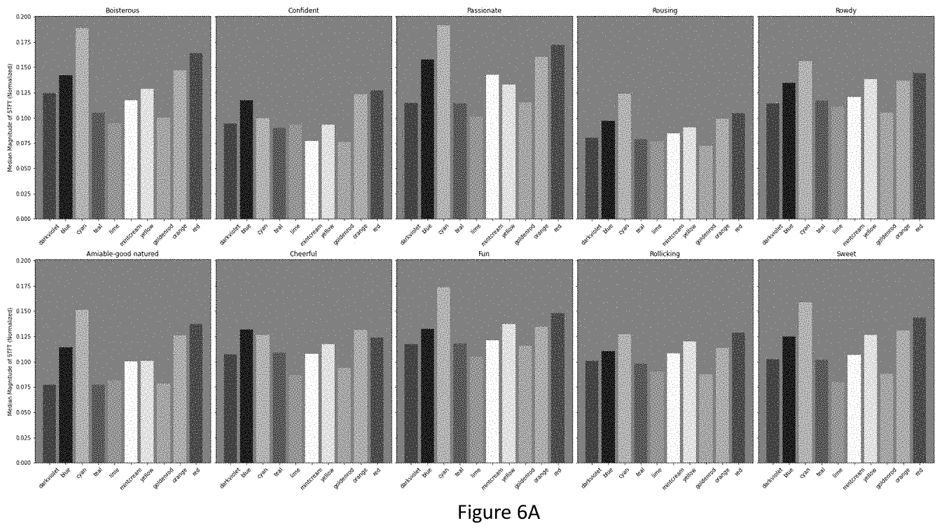

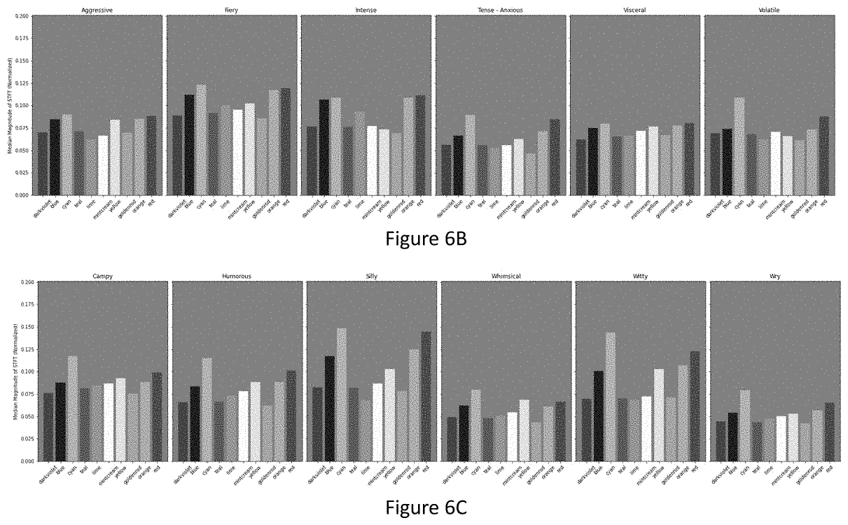

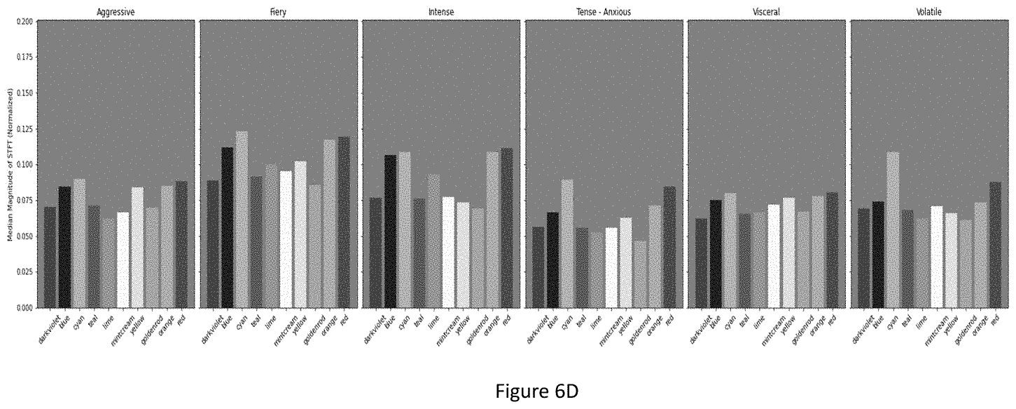

Data can be received as an image and color categorization. U4Ea analyzes the color content of an image, generating a histogram that reveals the proportionate ratios of colors in an image. Those ratios are reduced to a total impact of 1 with a U4Ea score inclusive of fractional contributions in multiple categories. Thus, visual files are aggregating each pixel in each frame (multiple frames for videos), creating a histogram of colors (e.g., FIGS. 5A, 5B) to be color-correlated (e.g., to FIG. 2). This can also be seen in FIGS. 6A-6D.

Data can also be received as audio and sound categorization. For example, in one embodiment U4Ea scores the number of times certain notes are played in a song (e.g., FIG. 4). This score reflects the number of times a note could have potentially triggered a subconscious response. Aggregated scores are reduced to a total impact of 1 with a U4Ea score inclusive of fractional contributions in multiple categories. Textual files are scored by aggregating the words used in the media, either spoken or written, color-correlated to the emotional and descriptive words and their synonyms organized in FIG. 2.

Data can also be received as text & lyric or text categorization: For example, in one embodiment U4Ea's text analysis is used to analyze musical content's textual component. Aggregated scores are reduced to a total impact of 1 with a U4Ea score inclusive of fractional contributions in multiple categories, e.g., as in FIG. 5.

Third party data, wearable data, and other elements discussed herein are processed according to FIG. 2, correlating areas of impact (steps and respiratory rate) to color (e.g., green).

The system processes the media content items identified in the customer data to determine for each content item a multidimensional content impact score (e.g., through FIG. 1, Sentiment/Content Scoring 5). In one embodiment, scoring includes weighting measures. For example, as the U4Ea Score is totaled, initially with equal weighting to the U4Ea App Data Sentiment Scoring and the External Data Sentiment Content Scoring, a final tally is provided, URE and the customer are informed of the customer's current “balance.” As depicted in FIG. 7, the net score for each of the seven categories is represented by a dot, some distance from the center line, indicative of balance, revealing the extent of their positive or negative score. The color-correlated (media) data gathered will be scored according to FIG. 8, granting one point to each of the seven colors (red, orange, yellow, green, bule, indigo, and violet (ROYGBIV).

Media files with lots of content are expected to score very high tallies. Each media score will be reduced to a total value of 1 with fractions of each color summing to a value of 1 for the total impact of a unit of media. The value of that rating will be saved in the U4Ea database for future recommendations (e.g., FIG. 1 element 8).

Within FIG. 2, columns are marked positive and negative. Contributions data correlated with negative impacts will receive a score of negative (−) 1.

As seen in FIG. 1 the system can process, using a machine learning trained recommendation engine 6, the individual wellness score of the user and the content impact scores of the media items consumed by the user to generate a recommended set of wellness-mediating content items, the wellness-mediating content items comprising at least one of healing sounds, healing colors, healing words, and healing content.

As the machine learning trained recommendation engine (MLTRE) identifies which pieces of content are most impactful, MLTRE will learn to weigh which inputs are the most impactful to the customer. For example, equal or skewed weighting can be applied, as discussed herein.

Individually scored content will be aggregated for a point-in-time analysis of the customer's subconscious mood state, to determine an Aggregated U4Ea Score. MLTRE is further trained as customers' moods are tracked and measured against the estimated subconscious impact. As seen in FIG. 8, the individual wellness score for the customer is an aggregate of all of the captured impacts (each scored unit of media, plus data), resulting a point in time U4Ea Score for 7 different categories. In one embodiment, that aggregate score is more simply represented by FIG. 7. These figures also explain the goal of U4Ea, to help customers shift from negative to balanced, meaning that the negative dots shift to the center. Both positive and negative scores are tracked for future recommendations.

In some examples, the scores (e.g., FIGS. 7 and 8) reveal categories of negativity (e.g., purple, blue, orange, and yellow). These areas represent the categories where recommendations will be offered.

The U4Ea Recommendation Engine (URE) (e.g., FIG. 1, element 7) takes the current point-in-time U4Ea Score for the customer, identifies the negative categories, then finds recommendations for those categories based on the customer's tracked history, e.g., what gave them positive purple, blue, orange, and yellow scores in the past? What activities are associated with those colors (eating an acai bowl, getting fresh air, basking in the sun light, or painting) and out of those, which are likely to motivate the customer?

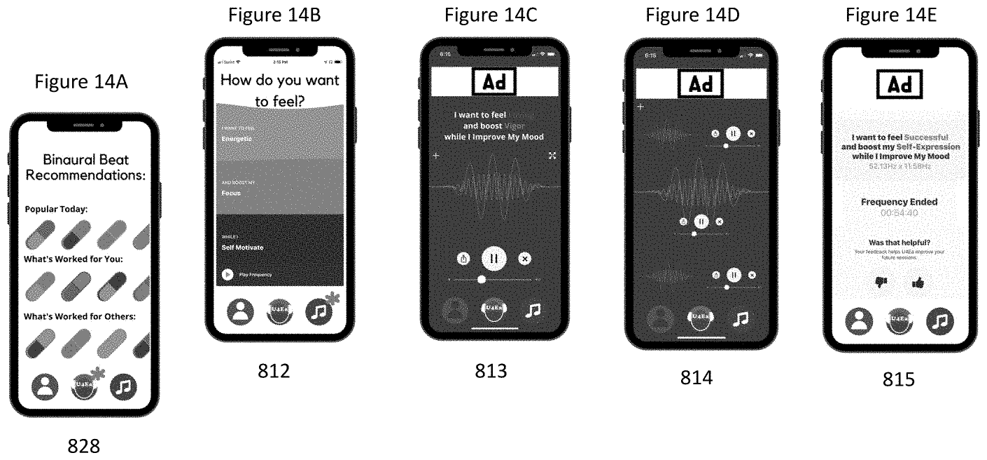

In one embodiment, the Recommendation Engine 500 (e.g., as shown in FIG. 511) and the efficacy of U4Ea (+80%) is built upon the recommendation of audio frequencies combined in a way to create a more desirable state of mind in an individual using a binaural beat. A binaural beat is an acoustic phenomenon that occurs when the vibrations of two different tones within 60 Hz of each other collide and create a third interference wave. The specificity of that wave along with the carrier tones creating it have yielded a range of wellbeing improving outcomes. Upon clicking on the U4Ea icon for Recommendations, the customer is brought to this page where they can scroll down to reveal a wider range of suggestions to improve their wellbeing. These options can be prioritized to be viewed above the scroll line, per the customer's settings and/or usage. The recommendation engine identifies which category of the U4Ea Score is most negative, pulls preference and activity information from the customer's profile, and suggests binaural beats (e.g., as shown in display 828 of FIG. 14A). According to FIG. 2, there are a number of modalities offering aligned categorization, from colors, nature, food, yoga positions, breathing exercises, media content and other alternatives. U4Ea's intent for web 3 is to provide access to these solutions as readily as we provide our own, thus: Recommendations are prioritized according to the customers' contributed and collected data.

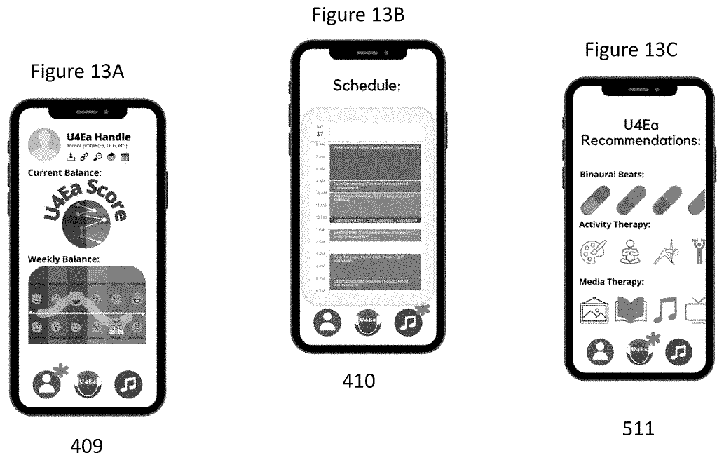

There are two components to the recommendation engine example depicted in FIG. 13C. Component 1 is U4Ea's Recommendation Engine (URE). The customer is given their U4Ea Score, indicating categories that may benefit from balancing. URE will suggest binaural beat combinations that counter the captured negative impacts previously analyzed. URE will also analyze the customer's preferences and past choices in order to recommend other modalities, such as breathing or humming, other products, and or other services.

Component 2 is U4Ea's Machine Learning Trained Recommendation Engine (MLTRE), which is a sentiment prediction recommendation system. The recommendations, as presented in FIG. 2, can range from binaural beats to yoga postures, creative activities, and a range a meditation practices to various forms of media and augmentations to improve wellbeing. The choices selected by the customer are categorized as ‘Preferred Features’ and are recorded with the customer's preferences. These Preferred Features are also analyzed by the FEATURE SELECTOR within the MLTRE.

The Features Selector gathers data from occurrences similar the most recently selected one, enabling the MODEL TRAINER to do a comparable analysis for time of day, day of the week, day of the month, location, previous mood, and more in order to estimate how much the selected feature (or intervention) will move the customer into balance within the targeted categories. In some preferred embodiments, the model trainer would run infinitely in order to home in on an accurate recommendation engine for each individual. In other embodiments, technical and economic rationale will dictate the number of model trainers available.

Once feedback from the intervention is gathered, it is analyzed in comparison to the model trainers within the WELLBEING OPTIMIZER. Here, the model trainer that most accurately predicted the customer's outcome will be qualified as the TRAINED U4Ea SCORE for integration into the next recommendation for the customer.

MLTRE tracks which selection the customer makes, correlates their choice to past choices, the choices of other users, and any other statistically relevant comparisons that enable an impact prediction, then track their generated or automatically-tracked biofeedback (e.g., FIG. 1, element 13) as discussed herein.

The accuracy of the impact prediction compared to the customer feedback continues to train MLTRE. MLTRE, once used, can be incorporated throughout the customer's next INPUT and experience, making (e.g., FIG. 1, element 9) an output on an electronic interface for the user of the set of recommended wellness-mediating content items generated by the MLTRE.

URE provides a scrolling list of modalities as a recommended set of wellness-mediating content items (as discussed herein) from binaural beats to yoga postures to media content. URE is also recommending media content based on its library of scored content that are in alignment with the recommended colors (books, articles, tv shows, and movies on matters of spirituality, communication, will power, and comedy). URE is also configured to recommend a host of tech-based augmentation that integrate positive use of colors and text.

Relative to FIG. 1 element 10, the method can further include outputting by causing at least one of the wellness-mediating content items from the set of wellness-mediating content items to be output to the user, for example in the form of binaural beats.

In some embodiments, the output can include a binaural beat generator that can be delivered as a wellness-mediating content item. For example, as shown in FIG. 14B, the Output (similar to, e.g., FIG. 10 Binaural Beats 800) is from a binaural beat generator. In some embodiments, this may be considered a core functionality, or in others an additional feature accessible only through additional unlocking of content or payment. In still other versions, this may be functionality accessible in the free section of the freemium offering. For example, customers can choose their combinations based on the lists provided. This page will appear blank and ready for inputs or pre-populated based on links and recommendations. Customers are prompted to answer three questions from this page: How do I want to feel? What attribute do I want to boost? And . . . What is my activity level? Pressing on each question takes the user to a page with listings of words to answer those questions. Those words are arranged according to U4Ea's Methodology. The answers indicate the combination of tones to be listened to through headphones or a speaker capable of bass level frequencies. Customers will be able to create their own combination of effects to experience. By answering three questions, how they want to feel, what attribute they'd like to boost, and their desired level of activity, U4Ea will create a binaural beat inclusive of their desired effects.

As seen in FIG. 1, element 11, at least one wellness-mediating content item is output to the user by augmenting a content item with the at least one wellness-mediating content item. As discussed herein, the delivery of audio, visual, and textual augmentations are available to the customer via URE. This can include, for example, slowing the tempo of a song to make it induce sleep or editing a document for the use of negative words.

In one embodiment, as seen in FIG. 1 element 10, the output can include outputting a binaural beat included in the set of recommended wellness-mediating content items.

In one embodiment, an Experience display can be interacted with to confirm features. For example, as seen in FIG. 14C, display 813, customers can be brought to an Experience interface when they select a pre-populating combination (e.g., I want to feel Loving and boost Vigor while I Improve My Mood, where Loving, Vigor, and My Mood were selected from available options). Details regarding the selected combination, along with features for customizing the experience, sharing content, and more are found here. The customer can control the play and paus features, change the pitch, tone, patterns and more. The customer can share their combination via links with the share icon. The customer can stop their session with the x icon. The customer can control the volume of their session with the sliding scale. The customer can expand the screen and explore other visuals. The customer can click the plus sign to add additional binaural beat layers. In one example the binaural beat page will come with the three questions already answered. This page will allow them to simply press play and begin to listen to their selected binaural beat.

In further embodiments, the output can be augmented with a content item not in the set of selected recommended wellness-mediating content items. For example, this can be an audio augmentation included in the set of recommended wellness-mediating content items.

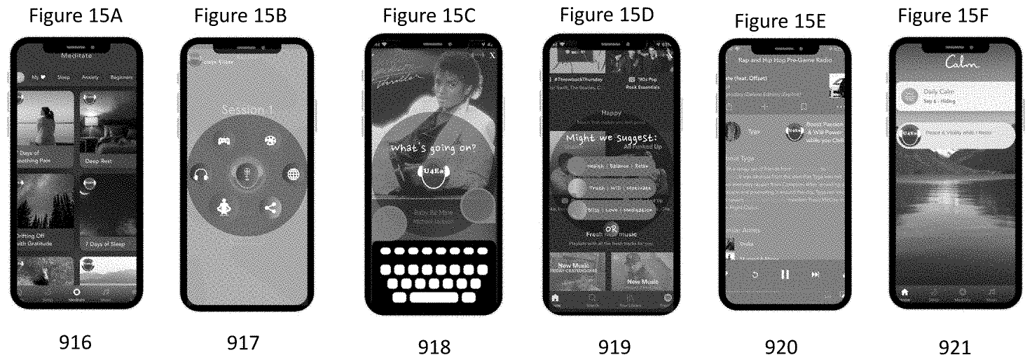

As seen in FIG. 1 element 11, audio/visual augmentations can take the form of integration with APIs and SDKs. Such functionality enables third parties to integrate U4Ea features into their platforms as in-app purchases, enabling U4Ea augmentation recommendations on those third party platforms based on the customer's current U4Ea Scores. For example, as shown in FIG. 15A display 916, as External features, U4Ea can scan content on third party platforms and make recommendations based on their content descriptions, providing opportunities to customize and enhance customer experiences.

In further embodiments, the augmenting can include augmenting with a content item not in the set of selected recommended wellness-mediating content items by a color augmentation included in the set of recommended wellness-mediating content items. Upon request, customers can integrate U4Ea features into experiences beyond the U4Ea platform. The below-discussed potential augmentations are not exclusive and described as a list of potential experiences that may be termed augmentations.

For example, relative to FIG. 1 element 11, URE can include audio/visual augmentations such as color augmentation recommendations that activate overlay screens that integrate current U4Ea Scores and external content to make augmentation recommendations on U4Ea and third party platforms. For example, as seen in FIG. 15A display 916, U4Ea can be augmented for external functionality, to scan content on third party platforms and make recommendations based on their content descriptions, providing opportunities to customize and enhance customer experiences. Overlay pop-ups can also be enabled, for example as seen in FIG. 15B display 917, U4Ea pop-up screens can be transparent overlays that provide menus for on-demand recommendations. U4Ea overlays function to shift the hue of the screen to assist in personal balance. Further still, as shown in FIG. 15C display 918, Notifications can be augmented to, using the overlay functionality, incorporate pop-up check-ins (as established by the customer) create opportunities for mindful reflection. Further still, as shown in FIG. 15D display 919, Responsive Recommendations can be augmented. As established by the customer, U4Ea can analyze ambient and/or media content as it's experienced, and offer recommendations via the platform or overlay pop-ups. Still further, as shown in FIG. 15E display 920, Media Recommendations can be augmented so that U4Ea can recommend songs, videos, articles, and other forms of digital media based on its U4Ea score and the customer's desired intention. As shown in FIG. 15F display 921, Targeted Modalities can be augmented so that U4Ea can recommend a spectrum of modalities from color dieting to yoga postures, to guided meditation on third party and/or customer-linked platforms (e.g., Calm, etc.).

In some embodiments, the system can play a piece of media content included in the set of recommended wellness-mediating content items.

For example, URE suggests media content based on the customer's preferences, the customer's tracked experiences, and the U4Ea Score on media units. Media content can be filtered for content the customer has access to, via the customer's owned content or content available via third party platforms, such as Spotify for music or YouTube for videos, etc. U4Ea can recommend songs, videos, articles, and other forms of digital media based it's U4Ea score and the customer's desired intention. This can be shown as, for example, in FIG. 15E display 920, Media Recommendations.

In FIG. 1, Item 5, sentiment or content scoring, the individual content score and the content impact scores can each include the dimensions. In one embodiment, analysis 400 is done through Scoring 400 (see FIG. 10). For example, there can be seven (7) categories of scoring, as detailed in FIG. 8, summarized in FIG. 7, and all based on the data in FIG. 2 to be applied to digital and media content wherein units of content are scored for impact, creating units of impact and content impact scores, which are recommended incorporated in URE and then measured against the customer's feedback with MLTRE. As the U4Ea Score is totaled, initially with equal weighting to the U4Ea App Data Sentiment Scoring and the External Data Sentiment Content Scoring, a final tally is provided, URE and the customer are informed of the customer's current “balance.” As depicted in FIG. 7, the net score for each of the seven categories is represented by a dot, some distance from the center line, indicative of balance, revealing the extent of their positive or negative score.

In determining sentiment or content scoring 5, the individual content scores and the content impact scores can each include a physical dimension, an emotional dimension, and a mental dimension.

The scores can be further broken down. For example, the U4Ea Score, the Physical, Emotional, and Mental (PEM) Scores can be broken down, and these scores can create further statistics like sensitivity, balance, and enthusiasm, which enable more links to recommendable products and services. To establish content not suitable for sensitive people or filter away balance-interrupting content will enable platforms to provide more options for sensitive customers to garner improved customer experiences. For example, as seen in FIG. 8, the U4Ea Score can be broken down into a PEM score: Physical, Emotional, and Mental. For example, the DaVinci interface from FIG. 4 will determine whether the customer is feeling pain from acute trauma or chronic pain, in either case gather information on the source of the pain being physical, or mental, or both. Aggregates of the PEM score can reveal more scores, like: Sensitivity: the total number of emotions tracked. Balance: ratio of total positive/neutral/negative scores. Enthusiasm: The average of the categorical scores. These scores can be used to create graphical representations used to inform the customer of their current and/or historical state(s). These scores are also used to inform the recommendation engine.

In other embodiments, in determining sentiment or content scoring 5, the individual content scores and the content impact scores can each include a physical dimension, an emotional dimension, and a mental dimension across seven different categories, each category corresponding to one of a color or an audio frequency range.

Correlations between (media) content and frequency are based on the U4Ea score of the content, which reflects the impact of that media, which is further made up by the physical, emotional, and mental influences tracked from that content, the color-correlated categories those results land in, and the frequencies associated with those corelated colors, based on FIG. 2. Thus a recommended binaural beat will take into account up to three negative categories, as determined by URE.

In additional embodiments, relative to FIG. 1 element 13, the method further includes electronically receiving biofeedback such as user feedback. The feedback can include the identification of at least one item of recommended wellness-mediating content consumed by the user and an impact on the physical, emotional, or mental well-being of the user resulting from such consumption.

U4Ea can, through various means, evaluate the efficacy of its recommendation engine. In one form, it takes a clinical approach to evaluating the efficacy of its recommendation engine. Once the customer has had some experience or is complete with their intervention, the customer will be prompted to reflect on their experience and with a thumbs up or down, emojis, a journal entry, or other tracked biofeedback, as like with a wearable device. For example, as seen in FIG. 14E display 815 and FIG. 10 Biofeedback 1100, data can be received as biofeedback. Biofeedback can come in the form of responses and inputs on the U4Ea platform and data from third party websites, platforms, apps, devices, etc. Data is analyzed according to U4Ea's mapped correlations to the endocrine system (i.e. Heart Rate Variability for respiratory analysis is categorized as green), identifying ratios of which categories were used most often. The ratios are again scored according to the seven point U4Ea Methodology, e.g., as shown in FIG. 2. After the customer has completed their experience with a recommended or selected intervention or after some time has passed while they are still experiencing their intervention, the customer is prompted to answer if U4Ea has helped them reach their targeted state of being. The customer can respond with a thumbs up or thumbs down. The customer can respond with a journal entry. The customer can respond using the DaVinci interface. The customer can respond using emojis. The customer's selections are categorized according to the below Data Categorization methodology in order to inform future recommendations.

In another embodiment, as seen in FIG. 14E display 815, U4Ea's objective is to improve the accuracy with which it makes recommendations that improve wellbeing, thus each session ends with an opportunity to leave (bio)feedback regarding the session. Implementing clinical approaches to data collection and analysis will also substantiate U4Ea's case for become a prescribe-able. Upon clicking the x icon to terminate the session, the customer is offered a recap on their session and the opportunity to review it. The thumbs up and thumbs down icons are for U4Ea to identify the efficacy of the recommendation and experience for future recommendations.

Relative to FIG. 1 element 6, in other embodiments the method also includes applying machine learning, such as a machine learning algorithm to adjust the processing of the recommendation engine for the user, a group of users, or all users, based on the feedback.

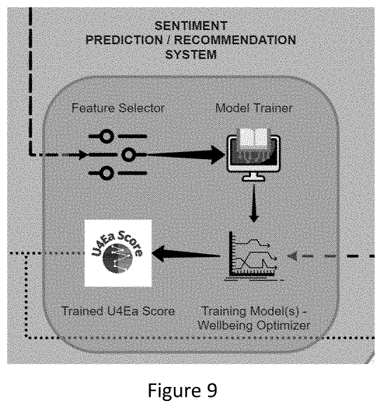

In some embodiments, feedback is provided, for example by closing the loop on the customer experience by integrating the customer's feedback on their selected intervention into MLTRE (e.g., as shown in FIG. 9). Platform content and other biofeedback and/or experience related data is integrated into the customer's profile (e.g., as shown in FIG. 6) to inform future recommendations. For example, if the customer indicated a positive shift in mood as the result of their change in location, binaural beats they listened to, a suggested book and a food recommendation, U4Ea will consider similar suggestions the next time mood symptoms are identified. Alternatively, U4Ea may recognize that certain customers respond better to variety or randomness, and will adapt accordingly.

MLTRE has already captured the selected intervention, the model trainer has predicted the expected impact, meaning, how far does MLTRE expect the intervention to move the indicators in a positive direction, and now the biofeedback is revealing the accuracy of the prediction. Successful predictions, as to be determined with ever improving standard deviations, will be used to introduce predicted options into the customer's selection choices (for emojis and recommendations), creating another opportunity for verifying the accuracy of U4Ea's recommendations. The ability to make predictions at the individual customer level will allow MLTRE to offer similar suggestions to similar people, based on their multiple factors of the customer profile. It is intended that this approach leads to more accurate recommendations for greater populations of people with less and less customer information required.

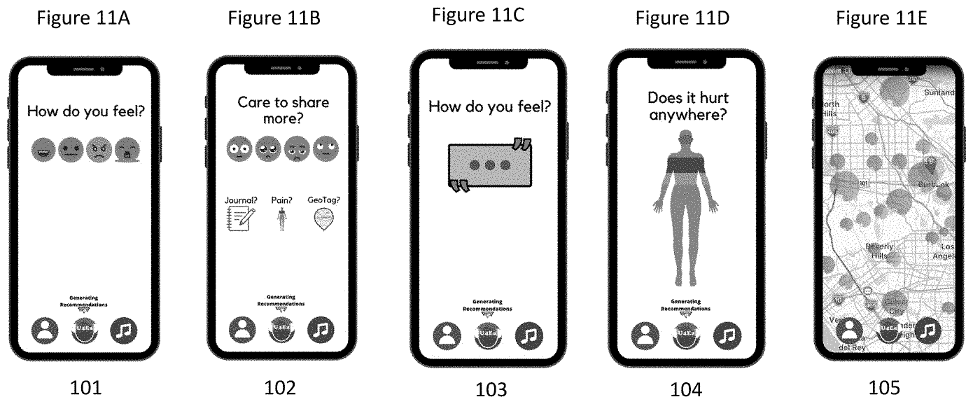

For example, one form of enhancing customer data can be achieved by layering sentiment information, which allows U4Ea to analyze patterns in behavior and to refine its recommendations. The intent is with more information to calculate, the more accurately the recommendation engine can make suggestions. Within U4Ea's categorical methodology, the more emotions that are expressed, the more accurately U4Ea can assess. Enhanced information can come in the forms similar to what was mentioned in (the previous comment), as well as other conceptual approaches. The results of the first question will prompt this second question, populating the interface with two emojis that might more accurately represent the customer, along with two other suggestions provided at random. Once the customer has gone through the process once, the fourth emoji suggestion will be suggested by U4Ea's MLTRE. Relative to, for example, FIGS. 11A and 11B, if the customer chooses the emoji circled in FIG. 11A, then FIG. 11B represents a possible configuration for allowing the customer to refine their input. There is the potential that the customer can refine their input up to six (6) or more times. Each of their submissions contributes to their U4Ea Score, a sentiment score. Once the customer is satisfied with their refinement, they can select the U4Ea icon for their recommendations (e.g., as in FIG. 511). Other means for capturing input from the customer include and are represented with icons for Journaling, Pain Recognition, and Habit Tracking.

In another example, as seen in FIG. 11C, the system includes Data Captured from Journal Input with Natural Language Processing. With U4Ea's library of emotional words, U4Ea can categorize the words used by individuals. U4Ea's text analysis will be trained to recognize dialect-based intention. For now, U4Ea measures the energetic confluence of their statement based on word count. When the customer selects the Journaling icon, Customers are prompted to answer questions such as, “How do you feel?” in their own words. Other solutions will involve selecting keywords. Once the customer submits their content, their answers will be analyzed by searching for specific words within a body of text. It then scores the body of text according to a synonym-based word list branching from FIG. 2 that categorizes emotional words, their synonyms, etc. Each word will add a single point tally for the appropriate category, adding to or initiating the U4Ea score, FIG. 7.

For example, as seen in FIG. 11D, data can be captured from the “DaVinci” input. When the customer selects the “Da Vinci” icon, the customer is brought to this interactive image where they are instructed to “Point to the Pain”. This information will inform the recommendation engine. The customer can submit multiple pain points, can elaborate on their pain with journaling, and more. Facilitated by the customer pointing to their (chronic) pain, U4Ea will be able to suggest with a high probability some potential sources for that pain as well as a variety of potential solutions, ranging from binaural beats and colors to media content. Each section a customer selects will add a single tally to their U4Ea score. This can be seen, for example, in FIG. 7.

In another example, as seen in FIG. 11E, the system includes Sentiment Mapping, and/or Data Captured from Geo-Tagging Input. Geo tagging will allow a customer to record how they feel and where they felt it. Allowing users to track their experiences can aid them in finding pleasurable options for balancing their mental, emotional, and physical states. Mapping/tracking such data will allow the customer to revisit positive experiences and avoid reliving negative ones. Once the customer settles on a description that fits their current situation, a bubble will populate on a map revealing their current location. They will also be able to view an aggregate map that reveals the culminated sentiment from other customers at locations on their map. Categorizing physical locations with emotional experiences allows U4Ea to suggest and reinforce positive opportunities for the individual and others with similar experiences, creating an opportunity for the customer to further curate their day to day.

In determining sentiment or content scoring 5, the identification of the impact on the physical, emotional, or mental well-being of the user can include a sentiment change value. For example, this can be as seen in FIG. 13A display 409.

Identification of the evidence that U4Ea is having a positive impact is reflected in the customer's profile page. This can be depicted as, for example, a Customer Profile as shown in FIG. 13A display 409. A summary page for a customer will allow them to view, analyze, and augment their activity on the platform. Their content (content they share with U4Ea) and their analysis, is kept private—while giving them the option to share. From this page are links to other features associated with the customer profile. The profile page will contain a chart highlighting their mood over time. The profile page will contain a chart indicating their current U4Ea score, revealing their current level of balance. The profile page will contain the three menu options for Home (returning you to the emoji screen to start a new “check-in”), the Recommendations button, and the BBB button. As the customer's experiences on U4Ea are tracked to inform recommendations, the profile page offers a summary of their current U4Ea Score and past scores. Visualizations will provide details revealing starting states of physical, emotional, and mental health, selected interventions and outcomes in the form of physical, emotional, and mental states after the intervention.

In other embodiments, the profile page will contain links to other features associated with the customer profile. These features can include, for example, Quick Analysis, Vision tracking, facial recognition, voice analysis, text analysis, scroll tracking, heart rate variability (HRV), electronic encephalogram (EEG) device analysis, and other app activity.

FIG. 11A shows data captured from emoji input. The goal of this screen is to initiate the building of a recommendation for the customer by obtaining information on how they are feeling. This screen offers the option to choose a representative emoji. Customers will select an emoji that represents their current emotional state. Each emoji is associated with an emotion as categorized by the chart in FIG. 2. The emoji the customer selects will add a single point tally for the appropriate category, starting the U4Ea Score, FIG. 7. There will also be an option for the customer to expand the menu and select an emoji themselves.

The customer has three other “Menu Options” represented by the “Profile” icon, the U4Ea Logo icon representing “Recommendations,” and U4Ea's foundational “binaural beat builder” (BBB), represented by the musical note icon. The Profile icon takes the customer to their private profile page (e.g., as shown in FIG. 13A display 409). The U4Ea icon takes them to the recommendation page (e.g., as shown in FIG. 511). Of note, the suggestions on the recommendation page updates as the customer enters data into the platform. The Musical icon takes them to where they can build a binaural beat combo (e.g., as shown in FIG. 14B display 812 with various options).

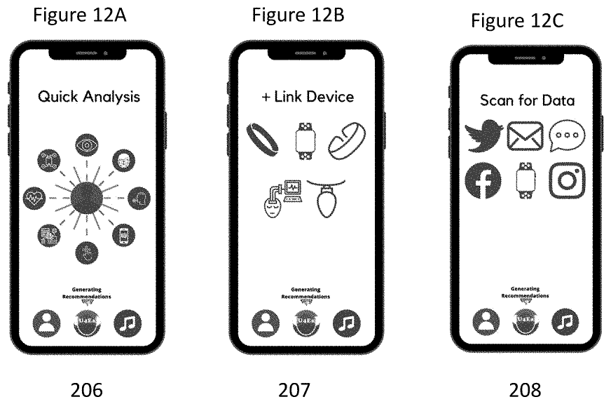

In other embodiments, the system includes a Quick Analysis. See, e.g., FIG. 10 element 206, or FIG. 12A. A customer will be able to click on any of the content identifying icons in order to experience another type of analysis. All settings are controllable by the customer. All data is private, to the customer's and third party platform's discretion.

For example, in some embodiments, the system includes Vision tracking. See, e.g., FIG. 10 element 206, or FIG. 12A display 206 element A [eye icon], which follows the direction your eyes focus, pupil dilation, blink rate, and more in order to identify stress levels and behavior patterns. This data will be synthesized according to the U4Ea methodology (FIG. 2) and integrated into the recommendation engine and customer experience.

In other embodiments, the system includes Facial recognition. See, e.g., FIG. 10 element 206, or FIG. 12A display 206 element B) [face icon], which analyzes movement as the result of emotional expressions to identify stress levels and behavior patterns. This data will be synthesized and integrated into the recommendation engine and customer experience.

In other embodiments, the system includes Voice analysis. See, e.g., FIG. 10 element 206, or FIG. 12A display 206 element C [speech icon], which recognizes inflections in the acoustics of a customer's speaking voice to identify stress levels and behavior patterns. This data will be synthesized and integrated into the recommendation engine and customer experience.

In other embodiments, the system includes Text analysis. See, e.g., FIG. 10 element 206, or FIG. 12A display 206 element D [chat icon], which uses U4Ea's Methodology (FIG. 2) for categorizing word selection to identify stress levels and behavior patterns. This data will be synthesized and integrated into the recommendation engine and customer experience.

In other embodiments, the system includes Scroll tracking. See, e.g., FIG. 10 element 206, or FIG. 12A display 206 element E [pointing icon], which analyzes touch rates on smart screens to identify stress levels and behavior patterns. This data will be synthesized and integrated into the recommendation engine and customer experience.

In other embodiments, the system includes Heart-Rate Variability (HRV). See, e.g., FIG. 10 element 206, or FIG. 12A display 206 element G [heart/rhythm icon], such that devices can track changes in your heart rhythm to identify stress levels and behavior patterns. This data will be synthesized and integrated into the recommendation engine and customer experience.

In other embodiments, the system includes Electronic Encephalogram (EEG) features. See, e.g., FIG. 10 element 206, or FIG. 12A display 206 element H [scanning device icon], such that devices can track changes in your brainwave patterns to identify stress levels and behavior patterns. This data will be synthesized and integrated into the recommendation engine and customer experience.

In other embodiments, the system includes Other-app Activity. See, e.g., FIG. 10 element 206, or FIG. 12A display 206 element F [network icon], which relates to data pulled from sources not owned by U4Ea, where the above mentioned quick analysis tools are used for analysis to identify stress levels and behavior patterns. This data will be synthesized and integrated into the recommendation engine and customer experience.

In other embodiments, the system includes Polyamorist Connections or Data Captured from Wearables. See, e.g., FIG. 10 element 207, or FIG. 12B display 207 [409B link icon]. There are two ways data can be captured from wearables. Option 1 is for the wearable to have U4Ea functionality incorporated so that the scoring is done locally, via an in-app add-on. Scores that are transferred from wearables will be aggregated into an accumulating U4Ea Score, as in FIG. 7. Option 2 is for agreed-upon data to be collected, then interpreted. U4Ea's “backend” infrastructure will intake appropriate/agreed-upon data and categorize it using U4Ea's methodology to make recommendations. Customers will be able to link their data tracking devices to our data receptive analysis tools. Data sharing agreements will reflect the transfer of relevant information for U4Ea scoring. Once any necessary translation of the data is complete, the score will be categorized with the scored information returned and/or deleted. The resulting score, according to the methodology from FIG. 2, will then be added to the accumulating U4Ea Score, as in FIG. 7.

In other embodiments, the system includes Polyamorist Data or Data Captured from Third Party Services. See, e.g., FIG. 10 element 208, or FIG. 12C display 208 [409C magnifying glass icon]. Customers can choose to have their experiences analyzed to help them navigate undesirable experiences. By enabling U4Ea to track the sound, light, color, text, shape and pattern beyond U4Ea's platform, customers can be informed of the subconscious influences that are affecting their mood, and thus, genetic expression. Data can be extracted from websites, app screens, videos, images, songs, and more. These features are largely available via APIs and partnership agreements enabling for streamlined integration and data processing. U4Ea will integrate the APIs such that the third party's native analysis will be correlated to U4Ea's Methodology in FIG. 2. third party analysis will inform the U4Ea score, adding to any accumulating U4Ea Score, FIG. 7. Should the third party lack sufficient analysis tools, U4Ea's API can integrate with their platforms so that U4Ea's scoring methodology can be scored locally and transferred without transferring the customer's private data. Should the transfer of private information be the only option available, the customer will have to opt in to the necessary parameters.

In other embodiments the system includes Data Captured from Media, where the customer will have the opportunity to have their media scored in order to identify the potential subconscious impact of the media, as well as recommending media that can have a positive impact. To do this, U4Ea analyzes sound, color, and text as discussed herein.

In other embodiments the system includes Data Captured from Customer Selection, where as the customer makes selections throughout the platform, U4Ea is tracking their “preferences” in order to provide optimal future recommendations.

In some embodiments, the system includes Data Categorization 300 (e.g., FIG. 10). The process of categorizing inputs and mapping them can be similar to that described as to FIG. 2, where U4Ea's methodology blends color psychology with music theory. The emotional responses associated to colors allows us to mathematically correlate colors to audible tones or frequencies, geometric patterns, words associated with emotions, the endocrine system, foods, and more.

Additional features and options can be utilized for the scoring. For example, the system may include an Aggregated U4Ea Score so that individually scored content will be aggregated for a point-in-time analysis of the customer's subconscious mood state. MLTRE is further trained as customers' moods are tracked and measured against the estimated subconscious impact.

Further still, the system can include Positive/Negative Analysis, so that received content is also scored according to impact. As the analysis is refined to recognize intent throughout various sentence structures, measuring word usage associated with mood states reveal the probability of predicting hormone imbalances for users and other applications.

Other features that may be found in one or more embodiments are described below. For example, in FIG. 13B display 410, the feature of Scheduling [409E calendar icon] is provided, where mood tracking is overlayed with a calendar feature can reveal chronic behavior patterns, thus providing opportunities for cognitive reframing with schedule balancing and other tools. A customer will be able to tag meetings to identify energetic load (type of experience, i.e. +/− emotions, emotional tagging), allowing U4Ea to integrate activities into the matrix. Clicking any icon will take you to an affiliate page where greater detail on how to access that product or service, along with its integrations with U4Ea, will be made available.

Similarly, in FIG. 11E display 105 the feature of Mood Mapping is depicted, where geographic mood tracking overlayed to a map can identify opportunities and recommendations for intentional activities.

Other Enhancements are shown in FIG. 14D display 814, e.g., layering Binaural Beats and augmentations allows for further customization and multiplied benefits/effects. Upon clicking the plus icon previously mentioned, the customer is introduced to FIG. 25, a pop-up menu. By selecting the headphone icon, they are returned to FIG. 20 to produce another combination. Upon completion, they are returned to FIG. 22 to view their active playlist.

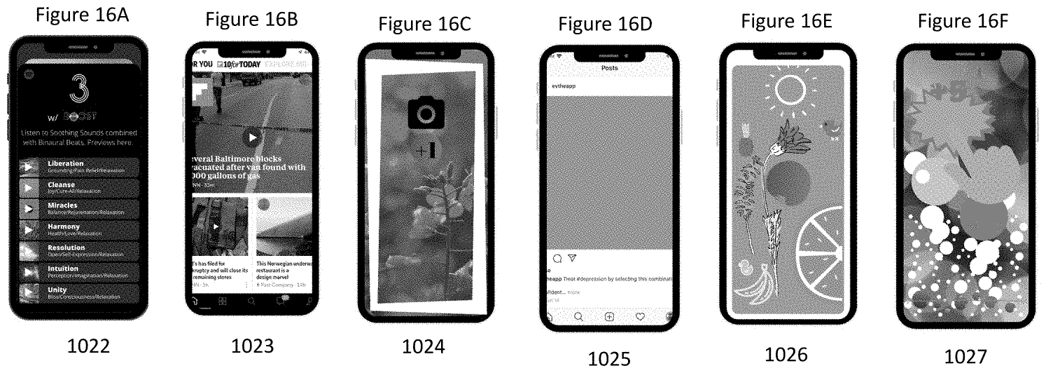

Further still, in FIG. 16A display 1022 an example of App Integration is shown where U4Ea's APIs allow external platforms to integrate U4Ea's binaural beat and sentiment recommendations. FIG. 16B display 1023 depicts an example of Content Scoring where U4Ea can provide indicators for the subconscious impact a piece of media may have on an individual. For example, letting a customer who is quick to anger know that there is inflammatory content in the news article he is about to read would provide them the opportunity to augment their experience with soothing binaural beats, a blue hued screen, and/or they can choose to read it as is, or ignore it.

Further still, in FIG. 16C display 1024 an example of Activity Therapy is shown, which enables recommendations on activities can range from listening to live music to sitting in the park. Other possible configurations include games involving photographing nature, i.e.—take pictures of everything you can find in nature that's yellow.

Several other options are also available. For example, Social Recycling (FIG. 16D display 1025) includes features where U4Ea can recommend public content that scores favorably for customers, creating new value in from old content. This is applicable to art, music, and social media posts, and accessible, scorable, digital content. Further, Cure-8tion (FIG. 16E display 1026) includes features where U4Ea can align artists with healing by providing a platform where U4Ea-scored art will be aggregated to create healing slideshows.

Further still, as seen in FIG. 16F display 1027, a feature of Game Therapy is provided as U4Ea incorporates its own designed interactive/educational games that empower individuals on their quests for greater mindfulness.

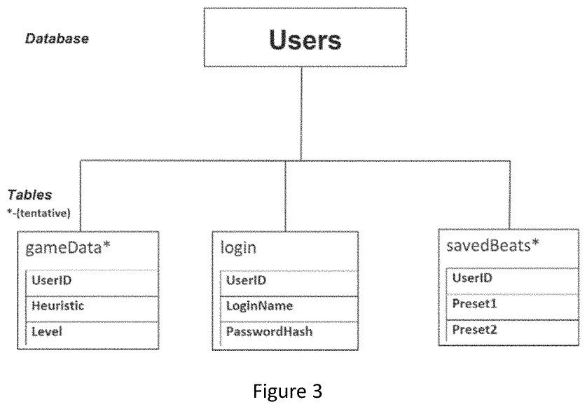

The system includes Data Storage or Local Storage, such as 700 shown in FIG. 10. In some examples, the ‘Customer's’ database consists of tables: Each row represents an entry to the table, in this table each row is a unique customer. There are 7 columns for the respective data for each customer. Data collected will be matched to the table in FIG. 13, assessing 1 point to each of the seven categories for each input it matches. In the ‘login’ table, each customer is designated: a unique UserID which is automatically generated by the table, a LoginName chosen by the user, and a PasswordHash (refer to Security of User Data). The evolutionary path for the database, with the gamification of the U4Ea using a heuristic score so each user can track their progress will look like e.g., FIG. 3. In one embodiment, as shown in FIG. 3, Connection with the Web App is facilitated. To move data back and forth from the app and the database you must first establish a connection. In the current web app, we are using JavaScript to configure the database in a file named config.js: The lines with the comment “update me” are dependent on the information on each individual database. Once there is a connection with the database, queries can be made by the file in the form of a “request”.

Security of User Data Storing: Customer data needs to be done with much forethought. Throwing every user's login information into one table poses a huge security risk. Therefore, we must do everything in our power to store this data in a way that is hard to access and furthermore, impossible to decipher. In most web browsers there is a tool called inspect element, anyone can use this by simply right-clicking on any website. This allows a client to be able to see the code that makes up the web page they are using. This is a great tool that can helps both the developer and the client, however, with lack of care this could leek sensitive information. To avoid this, we need to make sure the client does not have access to the files in the previous section (Connection with the Web App). These files should be held on the server side where data can be sent. This way the database information cannot be accessed. In addition, it is bad practice for the user password's to be explicitly stored anywhere. No one, including the company, should be holding on to this information. So instead of storing each user's password we should be storing a hashed (encrypted) version of the password. This hashing function will be nearly impossible to break if it is one-way encrypted. In one embodiment, proprietary or known encryption methods can be utilized. For example, JavaScript offers a library called bcrypt that offers a safe way to store passwords. This can provide a framework for generating a hash, registering a new customer, and comparing a raw and hashed password for logging in.

In one embodiment of the presently disclosed technology, a customer who has a social media platform can now grant their visitors a better filter to avoid, augment, and/or limit certain types of content based on predicted mental and emotional impact.

In one embodiment of the presently disclosed technology, a customer on a social media platform whose activity is scoring negatively (using anger as an example, the customer is in the negative red category) can have her screen shaded to a color (light red/pink/orange) to promote a positive shift in wellbeing (from anger to passion and creativity) as well as play audio designed around the octaves of 396 Hz.

In one embodiment of the presently disclosed technology, an individual showing an aversion to the color yellow, exhibits shy or anti-social behavior, expresses words associated with a lack in confidence and determination, all of which is supported by biofeedback data via wearables, phone/app activity, content consumed, and other data processed through U4Ea yields a score heavy in negative yellow. U4Ea will then prompt positive behavior associated with the color yellow to trigger parasympathetic responses in that region of the body with direct augmentation/suggestions (based on customer preference) ranging from sounds to listen to (via U4Ea's platform and others—emphasizing or augmenting to music designed around 528 Hz), yellow/golden shades to tint screens or lenses, media to consume (songs, movies, articles and more that score high in motivation, determination, courage) and media to avoid (content containing bullying, insecurities, depression), things to look for in nature (yellow flowers, bees, etc.) things to eat/drink (bananas, lemonade), and other suggestions associated with improving wellbeing. U4Ea then tracks, and later, checks in with the individual to see how the suggestions improved their day.

Various aspects of the subject matter described in this specification can be implemented in a computing system that includes a back-end component, e.g., such as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. The communication network can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

The computer system can include clients and servers. A client and server are generally remote from each other and typically interact through a communication network. The relationship of client and server arises by virtue of computer programs running on the respective computers and having a client-server relationship to each other. The computer system can be, for example, and without limitation, a desktop computer, laptop computer, or tablet computer. The computer system can also be embedded in another device, for example, and without limitation, a mobile telephone, a personal digital assistant (PDA), a mobile audio player, a Global Positioning System (GPS) receiver, a video game console, and/or a television set top box.

The term “machine-readable storage medium” or “computer-readable medium” as used herein refers to any medium or media that participates in providing instructions to the processor for execution. Such a medium may take many forms, including, but not limited to, non-volatile media, volatile media, and transmission media. Non-volatile media include, for example, optical or magnetic disks, such as the data storage device. Volatile media include dynamic memory, such as the memory. Transmission media include coaxial cables, copper wire, and fiber optics, including the wires that comprise the bus. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip, or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

The techniques described herein may be implemented as method(s) that are performed by physical computing device(s); as one or more non-transitory computer-readable storage media storing instructions which, when executed by computing device(s), cause performance of the method(s); or as physical computing device(s) that are specially configured with a combination of hardware and software that causes performance of the method(s).

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

To the extent that the terms “include,” “have,” or the like is used in the description or the claims, such term is intended to be inclusive in a manner similar to the term “comprise” as “comprise” is interpreted when employed as a transitional word in a claim. The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products. Other variations are within the scope of the following claims.

What is claimed is:

1. A method for identifying wellness-mediating content, comprising:
receiving customer data including user profile data and a plurality of media content items consumed by a user associated with the user profile data;
processing at least a subset of the customer data to determine a multidimensional individual wellness score for the user;
processing the media content items identified in the customer data to determine for each content item a multidimensional content impact score;
processing, using a machine learning trained recommendation engine, the individual wellness score of the user and the content impact scores of the media items consumed by the user to generate a recommended set of wellness-mediating content items, the wellness-mediating content items comprising at least one of healing sounds, healing colors, healing words, and healing content; and
outputting on an electronic interface for the user the set of recommended wellness-mediating content items generated by machine learning trained recommendation engine.

2. The method of claim 1, further comprising causing at least one of the wellness-mediating content items from the set of wellness-mediating content items to be provided to the user.

3. The method of claim 2, wherein the at least one wellness-mediating content items is provided to the user by augmenting a content item with the at least one wellness-mediating content item.

4. The method of claim 2, wherein the provision comprises one of:
outputting a binaural beat included in the set of recommended wellness-mediating content items,
augmenting a content item not in the set of selected recommended wellness-mediating content items by an audio augmentation included in the set of recommended wellness-mediating content items,
augmenting a content item not in the set of selected recommended wellness-mediating content items by a color augmentation included in the set of recommended wellness-mediating content items, and
playing a piece of media content included in the set of recommended wellness-mediating content items,

5. The method of claim 1, wherein the individual content score and the content impact scores each include the dimensions.

6. The method of claim 5, wherein the individual content scores and the content impact scores each include a physical dimension, an emotional dimension, and a mental dimension.

7. The method of claim 6, wherein the individual content scores and the content impact scores each include a physical dimension, an emotional dimension, and a mental dimension across seven different categories, each category corresponding to one of a color or an audio frequency range.

8. The method of claim 1, further comprising electronically receiving user feedback, the feedback including the identification of at least one item of recommended wellness-mediating content consumed by the user and an impact on the physical, emotional, or mental well-being of the user resulting from such consumption.

9. The method of claim 8, comprising applying a machine learning algorithm to adjust the processing of the recommendation engine for the user, a group of users, or all users, based on the feedback.

10. The method of claim 8, wherein the identification of the impact on the physical, emotional, or mental well-being of the user comprises a sentiment change value.
